(19) United States
(12) Patent Application Publication
(10) Pub. No.: US 2025/0230232 A1
(43) Pub. Date: Jul. 17, 2025

(54) IMMUNOLOGIC EFFECTOR CELL OF TARGETED CLD18A2, AND PREPARATION METHOD AND USE THEREOF

(71) Applicant: CRAGE medical Co., Limited, Hong Kong (CN)

(72) Inventors: Huamao Wang, Shanghai (CN); Bo Song, Shanghai (CN); Xiumei Cai, Shanghai (CN)

(21) Appl. No.: 18/806,496

(22) Filed: Aug. 15, 2024

Related U.S. Application Data
(63) Continuation of application No. 17/522,284, filed on Nov. 9, 2021, now Pat. No. 12,098,199.
Application No. 17/522,284 is a continuation of application No. 16/448,053, filed on Jun. 21, 2019, now Pat. No. 11,198,729.
Application No. 16/448,053 is a continuation of application No. 15/326,974, filed on Jan. 17, 2017, now Pat. No. 10,377,822.

(30) Foreign Application Priority Data
Jul. 17, 2014 (CN) 201410341504.X

(51) Int. Cl. C07K 16/28; A61K 35/12; A61K 35/17; A61K 40/11; A61K 40/31; A61K 40/42; A61K 48/00; C07K 14/705; C07K 14/725; C07K 19/00; C12N 5/0783; C12N 5/10; C12N 15/62; C12N 15/867

(52) U.S. Cl. CPC C07K 16/28; A61K 35/17; A61K 40/11; A61K 40/31; A61K 40/4202; A61K 48/00; C07K 14/7051; C07K 14/70517; C07K 14/70521; C07K 14/70578; C07K 19/00; C12N 5/0636; C12N 5/10; C12N 15/62; C12N 15/867; A61K 2035/124; A61K 2239/51; C07K 2317/53; C07K 2317/622; C07K 2319/02; C07K 2319/03; C07K 2319/70

(57) ABSTRACT

Disclosed are a chimeric antigen receptor (CAR) targeting CLD18A2, and preparation method and use thereof. The extracellular binding region of the CAR comprises a protein specifically recognizing CLD18A2. The immune effector cell modified by the CAR can be used to treat tumors such as pancreatic cancer and stomach cancer.

### CROSS REFERENCE

This application is a continuation application of U.S. patent application Ser. No. 17/522,284, filed Nov. 9, 2021, which is a continuation application of U.S. patent application Ser. No. 16/448,053, filed Jun. 21, 2019, now U.S. Pat. No. 11,198,729, which is a continuation application of U.S. patent application Ser. No. 15/326,974, filed Jan. 17, 2017, now U.S. Pat. No. 10,377,822, which is a national stage entry of International Application No. PCT/CN2015/084023, filed Jul. 15, 2015, which claims priority to Chinese Patent Application No. 201410341504.X, filed Jul. 17, 2014, each of which is entirely incorporated herein by reference.

### SEQUENCER LISTING

This application contains a Sequence Listing which has been submitted electronically in XML format. The Sequence Listing XML is incorporated herein by reference. Said XML file, created on Oct. 23, 2024, is named 63111-703_303_SL.xml and is 46,708 bytes in size.

### TECHNICAL FIELD

The invention belongs to the field of cell therapy for tumor, and particularly relates to an immune effector cell targeting CLD18A2, a preparation method and application thereof.

### TECHNICAL BACKGROUND

Attention has been increasingly paid to the role of T lymphocytes in tumor immune responses. The Adoptive immunotherapy based on T lymphocytes has a certain effect in some tumors., moreover, such immune therapy method can overcome the above defects of antibody treatment, however, the therapeutical effect in most tumors is still not satisfactory [Grupp S A, et al. Adoptive cellular therapy. Curr Top Microbiol Immunol., 2011; 344:149-72]. In recent years, based on the discovery that the identification of a target Cell by CTL is specifically dependent on a T lymphocyte receptor (T Cell receptor, TCR), the scFv of the antibody against tumor cell-related antigen is fused to intracellular signal activation motif such as T-lymphocyte receptor CD3ζ or FcεRIγ to form Chimeric antigen receptors (CAR), and can be genetically modified on the surface of T lymphocyte by means such as lentivirus infection. Such CAR T lymphocyte can selectively direct T lymphocyte to tumor cells and specifically kill tumor cells in a major histocompatibility complex(MHC)-independent manner. CAR T lymphocytes are new immune therapy strategy in the tumor immunotherapy field [Schmitz M, et al. Chimeric antigen receptor-engineered T cells for immunotherapy of Cancer. J Biomed Biotechnol, 2010, doi:10.1155/2010/956304].

Chimeric antigen receptor comprises an extracellular binding domain, a transmembrane region and an intracellular signaling domain. Generally, the extracellular domain comprises an scFv that is capable of recognizing a tumor-associated antigen, the transmembrane region employs the transmembrane region from molecules such as CD8, CD28 and the likes, and the intracellular signaling domain employs an immunoreceptor tyrosine-based activation motif (ITAM) CD3ζ or FcεRIγ and the intracellular signaling domain of co-stimulatory signaling molecule such as CD28, CD27, CD137, CD134 and the likes.

In the first generation CAR T lymphocyte, the intracellular signaling domain comprises ITAM only, and parts of the chimeric antigen receptor are connected in the form of scFv-TM-ITAM. Such CAR T can induce cellular cytotoxic effect against tumor, but the level of cytokines secreted is relatively low, and no sustaining anti-tumor effect could be induced in the body (Zhang T. et al., Chimeric NKG2D-modified T cells inhibit systemic T-cell lymphoma growth in a manner involving multiple cytokines and cytotoxic pathways, Can Res 2007, 67 (22): 11029-11036).

In the second generation CAR T lymphocyte that developed afterwards, an intracellular signaling domain of CD28 or CD 137 (also known as 4-1BB) is further included, and parts of the chimeric antigen receptor are connected in the form of scFv-TM-CD28-ITAM or scFv-TM-/CD137-ITAM. Co-stimulatory effect of B7/CD28 or 4-1BBL/CD137 in the intracellular signaling domain induces sustained proliferation of T lymphocytes, and is capable of increasing the level of cytokines such as IL-2, IFN-γ and others secreted by T lymphocytes, as well as improving the in vivo survival period and the anti-tumor effect of the CAR T (Dotti G. et al., CD28 costimulation improves expansion and persistence of chimeric antigen receptor modified T cells in lymphoma patients. J Clin Invest, 2011, 121 (5):1822-1826).

In the third generation CAR T lymphocyte that developed in recent years, parts of the chimeric antigen receptor are connected in the form of scFv-TM-CD28-CD137-ITAM or scFv-TM-CD28-CD134-ITAM, the in vivo survival and the anti-tumor effect of the CAR T is further improved (Carpenito C, et al., Control of large established tumor xenografts with genetically retargeted human T cells containing CD28 and CD 137 domains, PNAS, 2009, 106(9): 3360-3365).

Besides the attractive prospect of CAR T lymphocyte in tumor immunotherapy, its relatively high risk shall be taken into account. For instance, certain normal tissue(s) may exhibit low expression of specific antigen to be recognized by the CAR, this may results in damage by CAR T lymphocytes to such normal tissues. For example, treatment against carbonic anhydrase IX (CAIX), the antigen expressed in tumor cells of patients having renal cell carcinoma, is the first reported case of clinical application of adoptive therapy with CAR T lymphocytes, which is also the first case reporting on-target off-tumor effect of CAR T lymphocytes. After multiple administrations of CAR T lymphocytes, patients developed liver toxicity of grades 2-4. Upon analysis, the cause is believed to be the CAIX expression in a low level on bile duct epithelial cells, this clinical trial was discontinued while assessment about therapeutic outcomes in patients are excluded (Stoter G. et al., Treatment of metastatic renal cell carcinoma with autologous T-lymphocytes genetically retargeted against carbonic anhydrase IX: first clinical experience, J clin oncol, 2006, 24 (13): e20-e22; Ngo M C, et al., Ex vivo gene transfer for improved adoptive immunotherapy of cancer Human Molecular Genetics, 2011, R1_R7). Furthermore, the excessive co-stimulation signal in CAR may reduce the threshold required for activating effector cells, such that genetically modified T lymphocyte may be activated at conditions of rather low level of antigen or at the absence of antigen pulse, and resulting in the release of large amount of cytokines which may induce so-called “cytokine storm”. This signal leakage will cause off-target cytotoxicity, resulting in non-specific tissue damage. For example, sudden death of a patient caused by such “cytokine storm” induced by low Her2 expression in normal lung tissue was observed during a clinical treatment using a third-generation CAR T cells targeting Her2 for patients having advanced colorectal cancer with liver and lung metastasis (Morgan R A, et al., Report of a serious adverse event following the administration of T cells transduced with a chimeric antigen receptor recognizing Erbb2 Molecular Therapy, 2010, 18 (4): 843-851).

When CAR T is designed, selection of the antigen gene target is crucial. Because of the complexity of in-vivo gene expression and various uncontrollable factors, it is extremely difficult to select a suitable gene for CAR T. Furthermore, for many tumor-specific antigens, it is very difficult to find specific molecule directing at it and suitable to construct CAR-modified immune effector cell. After the CAR T is established, it is often unable to obtain an active extracellular binding region, which is also a difficulty for developing CAR T technology.

### SUMMARY OF THE INVENTION

The object of the present invention is to provide an immune effector cell targeting CLD18A2 and the preparation method and use thereof.

In a first aspect of the present invention, it is provided chimeric antigen receptor (CAR) expressed on the surface of an immune effector cell, wherein the chimeric antigen receptor comprises sequentially connected extracellular binding region, transmembrane region and intracellular signal region, wherein the extracellular binding region comprises protein which specifically recognizes CLD18A2 (claudin18.2).

In one preferred embodiment, the protein specifically recognizing CLD18A2 is an antibody or a ligand; preferably, the antibody is a single-chain antibody or a domain antibody.

In another preferred embodiment, the transmembrane region is a sequence comprising transmembrane regions and hinge regions of CD8 or CD28.

In another preferred embodiment, the intracellular signal region is selected from the intracellular signal region sequence of the following: CD3ζ, FcεRIγ, CD27, CD28, CD137, and CD134, and a combination thereof.

In another preferred embodiment, the chimeric antigen receptor comprises an extracellular binding region, a transmembrane region and an intracellular signal region connected in the following sequence:
Single chain antibody specifically recognizing the CLD18A2, CD8 and CD3ζ;
Single chain antibody specifically recognizing CLD18A2, CD8, CD137 and CD3ζ;
Single chain antibody specifically recognizing CLD18A2, transmembrane region of CD28(CD28a), intracellular signal region of CD28 molecule (CD28b) and CD3ζ; or
Single chain antibody specifically recognizing CLD18A2, transmembrane region of CD28, intracellular signal region of CD28, CD137 and CD3ζ.

In another preferred embodiment, the chimeric antigen receptor comprises any one of the amino acid sequence of SEQ ID NO: 19-22.

In another preferred embodiment, the immune effector cell comprises T lymphocytes, NK cells or NKT cells.

In another aspect of the invention, it is provided the nucleic acid encoding the chimeric antigen receptor.

In one preferred embodiment, the nucleic acid comprises any one of the nucleotide sequence of SEQ ID NO: 15-18.

In another aspect of the present invention, it is provided an expression vector comprising the aforementioned nucleic acid.

In one preferred embodiment, the expression vector is derived from lentivirus plasmid PWPT (or PWPT-eGFP).

In another aspect of the present invention, it is provided a virus, wherein the said virus (such as lentiviral vector) comprising said vector.

In another aspect of the invention, it is provided the use of the chimeric antigen receptor, the nucleic acid, the expression vector, or the virus, for preparing a genetically modified immune effector cell targeting CLD18A2.

In another aspect of the invention, it is provided a genetically modified immune effector cell transducted by said nucleic acid, said expression vector, or said virus.

In another aspect of the invention, it is provided a genetically modified immune effector cell wherein a chimeric antigen receptor is expressed on the surface thereof, wherein the amino acid sequence of the chimeric antigen receptor is selected from any one of the amino acid sequences of SEQ ID NOs: 19-22.

In another aspect of the present invention, it is provided the use of the genetically modified immune effector cells for preparation of a medicine for suppressing tumor, wherein the tumor is CLD18A2 positive (high-expression) tumor.

In another preferred embodiment, the CLD18A2 positive tumor includes pancreatic cancer, gastric cancer.

Other aspects of the invention will be apparent to those skilled in the art from the disclosure herein.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the structure schematic diagram of the present lentivirus vector pWPT-eGFP-F2A-CAR comprising CAR coding sequence.

FIG. 2 is a schematic diagram of the connection sequence of each part of the chimeric antigen receptor. “(G4S)3 linker” in FIG. 2 is set forth in SEQ ID NO: 26.

FIG. 3 is the electrophoresis image of the purified anti-CLD18A2 single chain antibody of Example 1.

FIG. 4 is the results of the western blot assay of the cell lines stably expressing CLD18A1 and CLD18A2.

FIG. 5 is flow cytometric detection of the binding specificity of CLD18A2 single-chain antibody with CLD18A1 and CLD18A2.

FIG. 6 is the electrophoresis identification of a spliced chimeric antigen receptor.

### DESCRIPTION OF THE PREFERRED EMBODIMENTS

Through thorough and in-depth study, the inventors discloses for the first time a CAR modified immune effector cell based on CLD18A2 gene and the preparation method thereof.

### CLD18A2 Gene

At early stage, the inventors investigated various kinds of tumor-specific genes and found that a relatively large part of these genes are also expressed in part of normal tissue cells, thus unable to be applied in the chimeric antigen receptor T cell. Some tumor-specific genes have better tumor-specific expression characteristics, but the CAR-modified immune effector cells correspondingly designed do not have or have rather low tumor cytotoxicity. This may be caused by that the proteins expressed by corresponding genes have low antigenicity, or expressed on an inapposite location, or expressed at a level not high enough, etc. It is also possibly caused by the recombinant construction process which weakened the tumor killing ability of T lymphocytes or cause the tumor killing ability lost.

After repeated investigation and screening, the inventors discovered CLD18A2 gene as the target gene for designing CAR modified immune effector cell (for example, T lymphocyte). Claudin 18 (CLD18) molecule (Genbank accession number: splice variant 1 (CLD18A1): NP.sub.-057453, NM.sub.-016369, and splice variant 2 (CLD18A2): NM.sub.-001002026, NP.sub.-001002026) is a transmembrane protein with a molecular weight of about 27.9/27.72 kD. Claudins is a tightly-connected membrane protein which locates on the epithelium and the endothelium.

Study shows that the CLD18A1 selectively expresses on the normal lung and stomach epithelium, while the CLD18A2 only expresses on differentiated cells with short lifespan in stomach epithelium, not on gastric stem cells. Meanwhile, researches have indicated that the CLD18A2 is expressed on various tumor cells. In view of the above-described characteristics of CLD18A2, the inventors have speculated CLD18A2 be an important therapeutic target for these tumors. Said speculation has been verified by abundant subsequent work.

### Chimeric Antigen Receptor and the Coding Nucleic Acid Thereof

The present invention provides a chimeric antigen receptor expressed on the surface of T lymphocyte, wherein the chimeric antigen receptor comprises sequentially connected extracellular binding region, transmembrane region and intracellular signal region, wherein the extracellular binding region comprises protein that specifically recognizes CLD18A2 (claudin 18.2). The chimeric antigen receptor is expressed on the surface of T lymphocyte, which make the T lymphocyte has highly specific cytotoxic effect on tumor cells which expressed CLD18A2 at a high level.

As a preferred mode of the present invention, the extracellular binding region comprises a single-chain antibody scFv that specifically recognizes CLD18A2 The extracellular binding region of the abovementioned chimeric antigen receptor protein is connected with a transmembrane region of CD8 or CD28 through a CD8 hinge region, and the cross-membrane region is immediately followed by the intracellular signal region.

The present invention also includes nucleic acid encoding the chimeric antigen receptors. The nucleic acid sequence of the present invention can be a DNA form or a RNA form. The DNA form comprises cDNA, genomic DNA, or artificially synthesized DNA. DNA can be single-stranded or double-stranded, a coding chain or a non-coding chain. The codons of the nucleic acid of the present invention coding the amino acid sequence of the present chimeric antigen receptor protein can be degenerate, that is, a variety of degenerate nucleic acid sequences encoding the same amino acid sequence are included in the scope of the present invention. The degenerate nucleic acid codons encoding corresponding amino acid are well known in the art. The present invention also relates to variants of the polynucleotide, which encode polypeptides or fragments, analogs and derivatives of the polypeptides having the same amino acid sequences as the present invention. The variants of the polynucleotide can be naturally occurring allelic variants or non-naturally occurring variants. These nucleotide variants include substitution variants, deletion variants, and insertion variants. As is known in the art, an allelic variant is an alternative form of a polynucleotide, which may be substitution, deletion or insertion of one or more nucleotides, but does not substantially alter the functionality of the polypeptide coded thereby.

The monoclonal antibody specifically recognizing human CLD18A2 can be chosen from the the antibodies disclosed in the prior art. A variety of monoclonal antibodies that recognize the c-terminal epitope of CLD18A2 may be applied in the present invention in a suitable manner, as long as after the recombinant construction, CAR-modified immune effector cell with killing activity can finally be obtained. Preferably, a single-chain antibody, more preferably the single-chain antibodies are 163 and 175 antibodies; The 163 and 175 antibodies can specifically recognize CLD18A2 but not CLD18A1. More preferably, they are connected to Fc (ScFv-163 and ScFv-175).

The term “single-chain antibody (scFv) fragment” as used herein refers to an antibody fragment defined as follows. It is a recombinant protein comprising heavy chain variable region (VH) and a light chain variable region (VL) connected by a linker, and the linker associates the two domains by which an antigen binding site is finally formed. The size of the scFv is generally ⅙ of one complete antibody. Preferably, single-chain antibody is one amino acid chain sequence coded by one nucleotide chain. Single-chain antibodies used in the present invention may be used alone or in combination with conventional techniques known in the art, for example, amino acid deletion, insertion, substitution, addition, and/or recombination and/or other modification methods for further modification. It is well known to those skilled in the art to introduce modification in the DNA sequence according to the amino acid sequence of the antibody, for example, see Sambrook, Molecular Cloning: A laboratory Manual, Cold Spring Harbor Laboratory (1989) N.Y. The modification is preferably performed on the nucleic acid level. The above single-chain antibodies can also comprise derivatives thereof. The “derivatives of antibodies” in the present invention include, for example, derivatives of the antibodies which obtained by phage display techniques, and the binding efficiency of said antibodies with CLD18A2 antigen epitope is increased by surface plasmon resonance technique that used in the Biacore system (Schier, Human Antibodies Hybridomas 7 (1996), 97-105; Malmborg «Journal of Immunological Methods», 1995, 183:7-13). Also included are those antibody derivatives produced by the preparation method of chimeric antibody described in for example WO89/09622, the humanized antibody preparation method described in EP-A10239400 and WO90/07861, the method for producing xenogeneic antibodies, for example human antibodies in mice, which is mentioned in WO 91/10741, WO 94/02602, and WO 96/33735.

The term “specific recognition” of the present invention means that the antibody of the present invention does not react with or substantially does not react with any polypeptide other than a target antigen. The degree of specificity can be determined by immunology techniques including, but not limited to, immunoblotting, immunoaffinity chromatography, flow cytometry and the like. In the present invention, the specific recognition is preferably determined by flow cytometry. Under particular conditions, the standard of specific recognition can be determined by those of ordinary skill in the art based on their knowledge of the art.

The transmembrane region of the chimeric antigen receptor can be selected from the transmembrane region of proteins such as CD8 or CD28. CD8 or CD28 are the natural markers on the surface of T lymphocyte. Human CD8 protein is a heterodimer, consisted of two chains, αβ or γδ. In one embodiment of the invention, the transmembrane region is selected from the transmembrane region of CD8 alpha or CD28. Additionally, the CD8 alpha hinge region (hinge)) is a flexible region. Therefore, the transmembrane region of CD8 or CD28 and the hinge region can be used to connect the target recognition domain scFv with an intracellular signal region in the chimeric antigen receptor (CAR).

The intracellular signal region may be selected from the intracellular signal regions of CD3ζ, FcεRIγ, CD28, CD137 and CD134 proteins, and combinations thereof. The CD3 molecule consists of five subunits, wherein CD3ζ subunit (also called as CD3 zeta, for short “Z”)) comprises a 3-ITAM motif, which is an important signal transduction region in TCR-CD3 complex. CD3δZ is a truncated CD3ζ sequence without ITAM motif, which is commonly used for constructing negative control in the practice of the present invention. FcεRIγ is mainly distributed on both mast cells and basophilic granulocyte surfaces, which contains an ITAM motif, and is similar to CD3ζ in structure, distribution and function. Furthermore, as previously described, CD28, CD137 and CD134 are co-stimulating signal molecules, which can cause sustained proliferation of T lymphocytes by the co-stimulation action generated by the intracellular signal segments after binding with the respective ligands, and can increase the level of cytokines secreted by T lymphocyte such as IL-2 and the IFN-gamma and the like, while increasing the survival cycle in vivo and the anti-tumor effect of the CAR-modified immune effector cells.

The anti-CLD18A2 chimeric antigen receptor protein coded by the nucleic acid of the present invention can be sequentially connected according to the following manner:
scFv(CLD18A2)-CD8-CD3ζ,
scFv(CLD18A2)-CD8-CD137-CD3ζ,
scFv(CLD18A2)-CD28a-CD28b-CD3ζ,
scFv(CLD18A2)-CD28a-CD28b-CD137-CD3ζ
and combinations thereof, wherein CD28a in the related chimeric antigen receptor protein represents a CD28 transmembrane region, CD28b represents intracellular signal region of CD28 molecules. The various anti-CLD18A2 chimeric antigen receptors above are collectively referred to as scFv (CLD18A2)-CAR.

In one embodiment of the invention, the nucleic acid disclosed by the invention has the sequence as shown in SEQ ID NO: 15-18. In another embodiment of the present invention, the nucleic acid of the present invention is a nucleic acid encoding the chimeric antigen receptor protein as shown in one of SEQ ID NOs: 19-22.

### Expression Vector and Cell

The present invention also provides vector comprising the nucleic acid encoding the abovementioned chimeric antigen receptor protein expressed on the surface of T lymphocyte. In one embodiment, the vector used in the present invention is a lentivirus plasmid vector pWPT-eGFP. Said plasmid belongs to a third-generation self-inactivated lentivirus vector system, which have 3 plasmids, that is, the packing plasmid psPAX2 encoding protein Gag/Pol and Rev protein; envelope vector PMD2.G encoding VSV-G protein; and empty vector PWPT-eGFP, which can be used for recombinant introduction of the target nucleic acid sequence, that is, a nucleic acid sequence encoding a CAR. In the empty vector pWPT-eGFP (which itself is mock in the following experiments), the elongation factor-1 alpha (EF-1α) promoter regulates the expression of the enhanced green fluorescent protein (eGFP), while in the recombinant expression vector encoding target gene CAR, the co-expression of eGFP and CAR is realized via ribosome skipping sequence 2A (shortened as “F2A”) from food-and-mouth disease virus (FMDV).

The present invention further comprises the virus comprising such plasmid. The virus disclosed by the invention comprises infectious virus after packing, as well as the to-be-packaged virus comprising essential element for packaging into an infectious virus. Other viruses known in the art for transferring foreign genes into T lymphocytes and their corresponding plasmid vectors can also be used in the present invention.

In one embodiment of the invention, the said virus is a lentivirus comprising the above-mentioned pWPT-eGFP-F2A-CAR recombinant vector (namely, comprising scFv (CLD18A2)-CAR).

The invention also provides a genetically modified T lymphocyte, which is transducted by the present nucleic acid, the present recombinant plasmid comprising the above mentioned nucleic acid, or the virus comprising the said plasmid. Conventional nucleic acid transduction methods in the present field, including non-viral and viral transduction methods, can be used in the present invention. The non-virus-based transduction method comprises electroporation method and transposition method. Recently, Nucleofector nuclear transfection apparatus developed by Amaxa can directly introduce foreign gene into the nucleus to realize high-efficiency transduction of target gene. In addition, the transduction efficiency of the transposon systems based on Sleeping Beauty system or PiggyBac transposon is greatly improved compared with that of common electroporation method, and it has already been reported about the combinative application of Nucleofector transfection apparatus and the Sleeping Beauty system (Davies J K., et al. Combining CD19 redirection and alloanergization to generate tumor-specific human T cells for allogeneic cell therapy of B-cell malignancies. Cancer Res, 2010, 70(10): OF1-10.) Such method not only has relatively high transduction efficiency but also can achieve site-directed integration of target genes. In one embodiment of the invention, the T lymphocyte transduction method for realizing the modification of the chimeric antigen receptor is based on virus such as retrovirus or lentivirus. The said method has advantages such as high transduction efficiency, stable expression of foreign genes, and can shorten the time for in-vitro culture of T lymphocytes to reach clinical scale. The transducted nucleic acid is expressed on the surface of the transgene tic T lymphocyte through transcription and translation. In vitro cytotoxity assay on various differently cultured tumor cells prove that the T lymphocyte modified by the present anti-CLD18A2 chimeric antigen receptor gene has highly specific tumor cell killing effect (also known as cytotoxicity). Therefore, the present nucleic acid encoding the chimeric antigen receptor protein, the plasmid comprising the said nucleic acid, the virus comprising the said plasmid and the transgenic T lymphocytes transducted by the above nucleic acids, plasmids or virus can be effectively used for tumor immunotherapy.

In one embodiment, the genetically modified T lymphocyte of the invention expresses a chimeric antigen receptor on the surface thereof, wherein the chimeric antigen receptor is coded and expressed by a nucleic acid of one of SEQ ID NOs: 15-18. In another embodiment, the transgenic T lymphocyte surface of the present invention expresses a chimeric antigen receptor, whose amino acid sequence is selected from one of SEQ ID NOs: 19-22.

Since currently there is no report of the CAR T targeting CLD18A2, the inventors for the first time successfully discovered an immune effector cell (eg, T lymphocyte) suitable for CAR modification from numerous tumor related genes, and successfully prepared CAR-modified immune effector cells. Thus, a brand new treatment means is provided for tumors such as pancreatic cancer and stomach cancer.

The embodiments of the present invention are further described below with reference to specific examples. It should be understood that these embodiments are for illustrative purposes only and are not intended to limit the scope of the invention. The experimental methods not specially noted of particular conditions are usually according to conventional conditions, such as those described in J. Sambrook et. al., Eds, Molecular Cloning: A laboratory Manual, 3'rd edition, Science Publishing House (2002), or the conditions recommended by the manufacturer.

### Example 1 the Expression of Single-Chain Antibody Against CLD18A2

By repeatedly researching and analyzing, the inventors identified several scFv antibodies recognizing CLDT8A2, for short, referred as 125,163, and 175.

125 (SEQ ID NO: 1 (nucleotide), 2 (amino acid)), 163 (SEQ ID NO: 3 (nucleotide), 4 (amino acid)), 175 (SEQ ID NO: 5 (nucleotide), and 6 (amino acid)) single-chain antibody sequences were synthesized by genetic synthesis based on bridging PCR. The synthesized products were digested by Nhe1/BamH1 (purchased from NEB), ligated in plasmid vector pCMV-V5-Fc (the said plasmid fuses and expresses human antibody Fc at the downstream of the multi-cloning site, hereinafter referred to as V5-Fc for short, purchased from Shanghai raygene biotechnology Co., LTD) digested by the same Nhe1/BamH1 via T4 DNA and was transformed into the host bacterium TOP10. The clones were picked and positive clones were identified by PCR, and confirmed by sequencing. V5-scFv-125-Fc, V5-scFv-163-Fc, and V5-scFv-175-Fc eukaryotic expression plasmid were obtained.

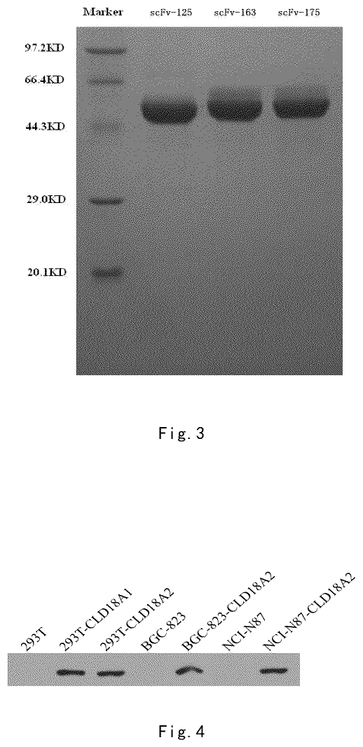

The above expression plasmids were used to transfect well grown HEK-293F, cultured at 37° C., 5% CO₂, 125 rpm on a shaking bed for 7 days. They were centrifuged for 10 min at 4000 rpm, and precipitate was removed. The supernatant were collected and filtered with a 0.45 μm filter film. The processed sample was purified by protein A affinity column (purchased from GE), and eventually the purified single-chain antibody-Fc fusion protein scFv-125-fc (for short scFv-125), scFv-163-fc (scFv-163 for short), scFv-175-fc (scFv-175 for short) were obtained. The identification result is shown in FIG. 3.

### Example 2. Construction of the Stable Expression Cell Line of CLD18A1 or CLD18A2

### 1. Construction of Expression Vectors of CLD18A1 and CLD18A2 and Preparation of Lentivirus

The complete coding sequences of CLD18A1 (GenBank: NM_016369) and CLD18A1 complete coding sequence (Genbank: nm_001002026) were synthesized by genetic synthesis technology based on bridging PCR. A flag tag(DYKDDDK (SEQ ID NO: 25)) was inserted into the c-terminal, and MluI/SalI (purchased from NEB) were added at both ends of the synthesized gene segments. The segments were double-digested by MluI/SalI, ligated in plasmid vector pWPT (purchased from addgene) double-digested by the same MluI/SalI via T4 DNA, and were transformed into the host bacterium TOP10. The clones were picked and identified by PCR, and confirmed by sequencing. That correct lentivirus vector plasmid PWPT-CLD18A1, PWPT-CLD18A2 were obtained. The above plasmids and packing accessory plasmids (pGag-pol, pREV, pVsv-g (all purchased from addgene)) were co-transfected at a certain ratio to 293T cell. After 48 h and 72 h of transfection, CLD18A1 and CLd18A2 virus solutions were collected, sub-packed, and stored at −80° C.

### 2. Establishment of Stable Exogenous Expression Lineage for CLD18A1 and CLD18A2 and Western Blot Assay

The above collected CLD18A1 or CLD18A2 virus solutions were added into 293T cells in 6 cm dish respectively. After 72 hours, cells were collected and were lysed by cell lysis solution. On the other hand, human stomach cancer BGC-823 (purchased from Shanghai cell library of Institution of Science of China, TCHu11) and NCI-N87 (purchased from ATCC, CRL-5822) were infected by CLD18A2 virus, respectively. After the cells grew to full, they were lysed by cell lysis solution. 40 μg protein from collected lysed cells were subjected to SDS-PAGE gel electrophoresis, and the gel was assayed by immunoblotting, stained with mouse anti-FLAG antibody (purchased from sigma Aldrich). After PBS wash, incubated together with goat anti-mouse antibodies labeled by horseradish peroxidase (purchased from santa cluz), and colored using ECL reagent, and finally, developed.

The Western blot results showed strips with molecule weight of about 28 kD in 293T cells transfected with CLD18A1 or CLD18A2 (i.e. 293T-CLD18A1, 293T-CLD18A2) and BGC-823 and NCI-N87 cells transfected by CLD18A2 (i.e. BGC-823-CLD18A2, NCI-N87-CLD18A2), but no strips in the untransfected empty cells (FIG. 4), indicating the successful construction of cell lines exogenously expressing CLD18A1 and CLD18A2.

3. Experiment Steps of Flow Cytometry Analysis of the Binding Profile of Each Cell Line with Anti-CLD18A2 Antibody

Using a fluorescence activated cell sorter (FACS) (BD company, FACSCalibur), the respective binding ability of single-chain antibodies scFv-125, scFv-163 and scFv-175 with each of the following cell lines were tested.

The specific method is as follows:

1) 293T, 293T-CLD18A1, 293T-CLD18A1, 293T-CLD18A2, BGC-823, BGC-823-CLD18A2, NCI-N87, NCI-N87-CLD18A2 tumor cells at exponential growth phase were inoculated into 6 cm flat dish with a inoculation cell density about 90%, and incubated overnight at 37° C. in incubator.

2) The cells were digested by 10 mM EDTA, and collected by centrifugation at 200 g×5 min. The cells were resuspended in 1% phosphate buffer solution containing calf serum (NBS PBS) at a concentration of 1*10⁶-1*10⁷/ml), and added into cytometric pipe at 100 ul/pipe.

3) Centrifuged at 200 μg for 5 min, and the supernatant were discarded.

4) The antibodies to be tested, scFv-125, scFv163 and scFv-175 were added, and simultaneously using unrelated antibodies as the negative control with final concentration of antibody of 20 μg/ml, and 100 ul of antibody in each pipe. Then they were left on Ice bath for 45 minutes.

5) Each pipe was added 2 ml 1% NBS PBS, centrifuged at 200 g for 5 min twice.

6) Supernatant was discarded. 1:50 diluted FITC-labeled goat anti-human antibody (from shanghai KangChen Bio-tech Inc), 100 ul per tube was added, and then put on ice bath for 45 minutes.

7) Each pipe was added 2 ml 1% NBS PBS, centrifuged at 200 g for 5 min twice.

8) The supernatant was discarded, and the pellet was resuspended in 300 ul 1% NBS PBS, detected by flow cytometry.

9) Flow Cytometer data analysis software WinMDI 2.9 was used to analyze the data.

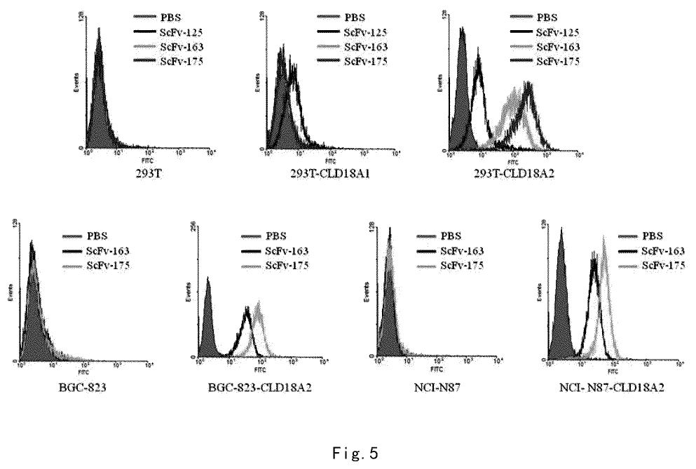

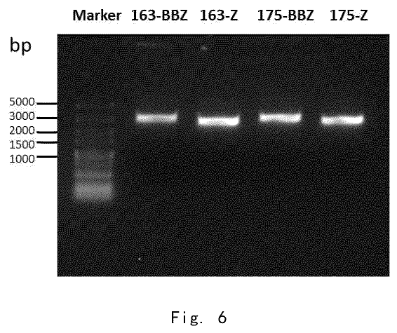

The flow cytometry results showed that the single-chain antibody scFv-125 can not only bind to CLD18A1 stably expressed 293T cells but also CLD18A2 stably expressed 293T cells (FIG. 5), indicating that this single-chain antibody lacks binding specificity for CLD18A2. Luckily, single-chain antibody scFv-163 and scFv-175 can specifically recognize the 293T stably expressing CLD18A2, not bind to 293T cells stably expressing CLD18A1, which indicates that these two single-chain antibodies can specifically recognize CLD18A2. Furthermore, these two single-chain antibodies can also specifically recognize BGC-823 or NCI-N87 cell lines stably transfected with CLD18A2, but do not bind to the BGC-823 or NCI-n87 cells not transfected with CLD18A2.

### Example 3 Construction of Lentiviral Plasmids Expressing Chimeric Antigen Receptor Proteins Encoded by the Nucleic Acids of the Present Invention, and Virus Packaging

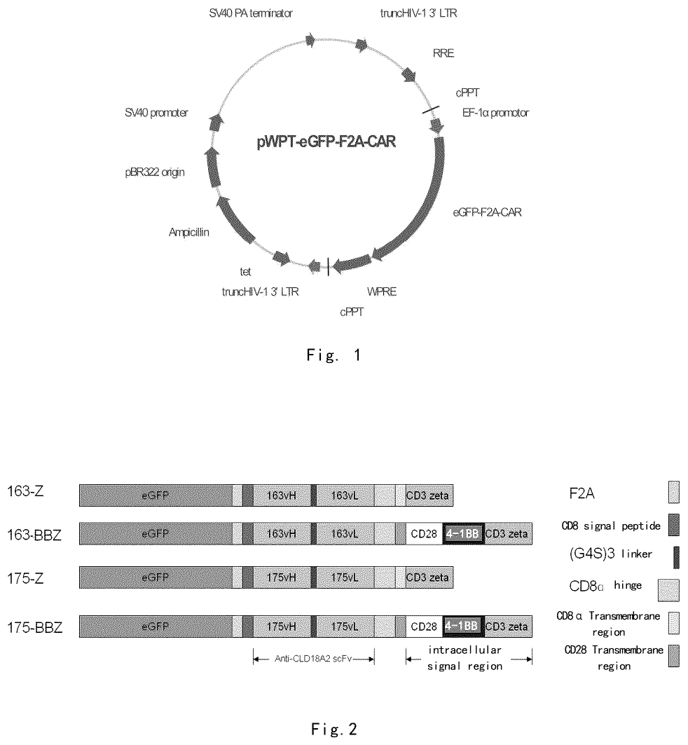

Table 1 explains the connection sequence of the exemplary chimeric antigen receptors of the present invention, the connection may also be seen in FIG. 2.

| | Extracellular binding region-transmembrane |
|---|---|
| Chimeric antigen | region-intracellular signal region 1- |
| receptor | intracellular signal region 2, etc |
| CLD18A2-δZ | scFv(CLD18A2)-CD8-CD3δzeta (negative |
| | control) |
| CLD18A2-163-Z | scFv(CLD18A2-163)-CD8-CD3 zeta |
| CLD18A2-175-Z | scFv(CLD18A2-175)-CD8-CD3 zeta |
| CLD18A2-163-28BBZ | scFv(CLD18A2-163)-CD28a-CD28b- |
| | CD137(i.e. 4-1BB)-CD3 zeta |
| CLD18A2-175-28BBZ | scFv(CLD18A2-163)-CD28a-CD28b-CD137- |
| | CD3 zeta |

### 1. Amplification of Nucleic Acid Fragments

(1) Amplification of scFv (CLD18A2-163, CLD18A2-175) Sequences

Using v5-scFv-163-fc plasmid as template, in the primer pair, the forward primer (SEQ ID NO: 7) comprises part of 2A sequence and the reverse primer (SEQ ID NO: 8) comprises part of CD8 hinge sequence. ScFv (CLD18A2-163) was obtained by PCR amplification. In the same way, using the v5-scFv-175-Fc plasmid as template, scFv (CLD18A2-175) was obtained by PCR amplification, using primer pair wherein the forward primer comprises part of 2A sequence (SEQ ID NO: 9) and the reverse primer (SEQ ID NO: 10) comprises part of the CD8 hinge sequence.

### (2) The Nucleic Acid Sequence of Other Parts of the Chimeric Antigen Receptor

Other part of anti-CLD18A2 chimeric antigen receptor protein except scFv(CLD18A2-163, CLD18A2-175) were obtained by PCR using the sequences SEQ ID NOs: 18 and 21 disclosed in CN 201310164725.X.

Wherein the eGFP-F2A sequence was obtained by PCR amplification using plasmid of SEQ ID NO:18 described in the patent application number 201310164725.X as template and SEQ ID NOs: 11 and 12 as primer pair.

Obtaining CD8-CD3ζ (Z) and CD28a-CD28b-CD137-CD3ζ (28BBZ): CD8-CD3ζ(Z) and CD28a-CD28b-CD137-CD3ζ(28BBZ) fragments were respectively obtained by PCR amplification using scFv(GPC3)-CD8-CD3ζ(SEQ ID NO:18 in patent application 201310164725.X) and scFv(GPC3)-CD28a-CD28b-CD137-CD3ζ (SEQ ID NO:21 in patent application 201310164725.X) as templates and SEQ ID NOs: 13 and 14 as primer pair.

SEQ ID NO: 18 in 201310164725. X is corresponding to SEQ ID NO: 23 in the present invention

The SEQ ID NO: 21 in 201310164725. X is corresponding to SEQ ID NO: 24 in the present invention.

### 2. Splicing of Nucleic Acid Fragments

The eGFP-F2A nucleic acid fragment obtained as mentioned previously, scFv (CLD18A2-163) or scFv (CLD18A2-175) nucleic acid fragments of equal molar, and CD8-CD3ζ (Z) or CD28a-CD28b-CD137-CD3ζ (28BBZ) nucleic acid fragments of equal molar were subjected to three-fragment splicing as shown in FIG. 2 and PCR. The splicing conditions were as follows: predenaturation at 94° C. for 4 min; denaturation at 94° C. for 40 s; annealing at 60° C. for 40 s; extending at 68° C. for 140 s, 5 cycles, and then overall extension at 68° C. for 10 min. After addition of DNA polymerase and forward primer (SEQ ID NO: 11) and reverse primer (SEQ ID NO: 14), the PCR amplification were done for 30 cycles, and the amplification conditions were: pre-denaturation at 94° C. for 4 min; denaturation at 94° C. for 40 s; annealing at 60° C. for 40 s; extension at 68° C. for 140 s, for 30 cycles; and then overall extension at 68° C. for 10 min. The fragments obtained after the amplification are as follows (Table 2):
eGFP-scFv(CLD18A2)-163-Z (SEQ ID NO: 15, 19),
eGFP-scFv(CLD18A2)-163-28BBZ (SEQ ID NO: 16, 20),
eGFP-scFv(CLD18A2)-175-Z (SEQ ID NO: 17, 21),
eGFP-scFv(CLD18A2)-175-28BBZ (SEQ ID NO: 18, 22).

The results identification where shown in FIG. 6.

TABLE 2
The sequences in the present invention

| Sequence | Description |
|---|---|
| SEQ ID NO: 1 | Nucleic acid sequence encoding CLD18A2 single |
| | chain antibody 125 |
| SEQ ID NO: 2 | Amino acid sequence of CLD18A2 single chain |
| | antibody 125 |
| SEQ ID NO: 3 | Nucleic acid sequence encoding CLD18A2 single |
| | chain antibody 165 |
| SEQ ID NO: 4 | Amino acid sequence of CLD18A2 single chain |
| | antibody 163 |
| SEQ ID NO: 5 | Nucleic acid sequence encoding CLD18A2 single |
| | chain antibody 175 |
| SEQ ID NO: 6 | Amino acid sequence of CLD18A2 single chain |
| | antibody 175 |
| SEQ ID NO: 7~14 | Primer sequences |
| SEQ ID NO: 15 | Nucleic acid sequence encoding chimeric antigen |
| | receptor protein CLD18A2-163-Z |
| SEQ ID NO: 16 | Nucleic acid sequence encoding chimeric antigen |
| | receptor protein CLD18A2-163-28BBZ |
| SEQ ID NO: 17 | Nucleic acid sequence encoding chimeric antigen |
| | receptor protein CLD18A2-175-Z |
| SEQ ID NO: 18 | Nucleic acid sequence encoding chimeric antigen |
| | receptor protein CLD18A2-175-28BBZ |
| SEQ ID NO: 19 | Nucleic acid sequence encoding chimeric antigen |
| | receptor protein CLD18A2-163-Z |
| SEQ ID NO: 20 | Amino acid sequence of chimeric antigen receptor |
| | protein CLD18A2-163-28BBZ |
| SEQ ID NO: 21 | Amino acid sequence of chimeric antigen receptor |
| | proteinCLD18A2-175-Z |
| SEQ ID NO: 22 | Amino acid sequence of chimeric antigen receptor |
| | protein CLD18A2-175-28BBZ |
| SEQ ID NO: 23 | scFv(GPC3)-CD8-CD3ζ (SEQ ID NO: 18 in |
| | 201310164725.X) |
| SEQ ID NO: 24 | scFv(GPC3)-CD28a-CD28b-CD137-CD3ζ (SEQ |
| | ID NO:21 in 201310164725.X) |

### 3. Lentiviral Plasmid Vector Construction Method

As an example, the vector system used for constructing the lentiviral plasmid vector of the present invention belongs to a third generation self-inactivating lentivirus vector system, which comprises three plasmids, namely, psPAX2 encoding Gag/Pol protein and Rev protein (purchased from addgene); envelope plasmid PMD2.G encoding VSV-G protein (purchased from addgene); and recombinant expression vector encoding target gene CAR which is based on empty vector PWPT-eGFP (purchased from addgene)

In the empty vector pWPT-eGFP, the elongation factor-1 alpha (EF-1α) promoter regulates the enhanced green fluorescent protein (eGFP), while in the recombinant expression vector encoding target gene CAR, the co-expression of eGFP and CAR is realized via ribosome skipping sequence 2A (shortened as “F2A”) from food-and-mouth disease virus (FMDV). F2A is a core sequence from FMDV 2A (or referred to as “self-splicing polypeptide 2A”), which has the “self-splicing” function of 2A, and can realize the co-expression of both upstream and downstream genes. Due to the high splicing efficiency, the highly balanced expression of upstream and downstream genes and the short sequence of itself, 2A provided an effective and feasible strategy for constructing polycistronic vector for gene therapy. In particular, this sequence is often used in the immune therapy based on chimeric antigen receptor gene modified T lymphocyte, to realize the co-expression of the target gene and GFP or eGFP, therefore CAR expression can be indirectly detected by detecting GFP or eGFP.

According to the present example, a lentivirus expression vector which co-expresses eGFP and a specific CAR linked by F2A was constructed, collectively, referred to as pWPT-eGFP-F2A-CAR (FIG. 1). The target gene eGFP-F2A-CAR obtained in step 2 (see 1(2) in Example 3, and the element following F2A is called as CAR for short) was digested by restriction endonucleases MluI and SalI, linked into a pWPT vector digested in the same way so as to construct a lentiviral vector expressing each chimeric antigen receptor. After the successfully constructed vector was confirmed by MluI and SalI digestive identification and sequencing, it can be subjected to lentivirus packaging. As previously mentioned, the eGFP-F2A-CAR was transcribed into a mRNA, but finally into two peptide chains as eGFP and anti-CLD18A2 chimeric antigen receptor. In the guidance of the CD8α signal peptide, the anti-CLD18A2 chimeric antigen receptor would locate on cell membrane.

The obtained vectors comprising target CAR are as follows:
pWPT-eGFP-F2A-scFv(CLD18A2)-163-Z;
pWPT-eGFP-F2A-scFv(CLD18A2)-163-28BBZ;
pWPT-eGFP-F2A-scFv(CLD18A2)-175-Z;
pWPT-eGFP-F2A-scFv(CLD18A2)-175-28BBZ.

### 4. Packaging Lentivirus by Plasmid Transfection of 293T

HEK-239T cells (ATCC: CRL-11268) cultured to 6-10 generations were inoculated at a density of 6×10⁶ in 10 cm petri dish, incubated at 37° C., under 5% CO₂ overnight, preparing for transfection. The culture medium was DMEM (purchased from PAA company) with 10% fetal calf serum (purchased from PAA company). The next day, the culture medium was replaced with serum-free DMEM at about 2 hours before transfection.

The transfection steps were as follows:
4.1 20 g empty plasmids pWPT-eGFP (mock control) or 20 μg individual target gene plasmid pWPT-eGFP-F2A-CAR, together with 15 μg of packaging plasmid PAX2 and 6 μg envelope plasmid pMD2.G were dissolved into 500 μL MillQ water, and uniformly mixed.
4.2 62 μL of 2.5M CaCl₂) (purchased from Sigma company) was added dropwise, and mixed homogenously at 1200 rpm/min vortex,
4. 3 Finally, 500 μL of 2×HeBS (80 mM NaCl, 10 mM KCl, 1.5 mM Na₂HPO4·2H₂O, 12 mM glucose, 50 mM Hepes (purchased from Sigma), pH7.05, sterilization with a 0.22 um filter) was added dropwise, and mixed homogenously at 1200 rpm/min for 10 s.
4.4 Immediately added into the petri dish dropwise, slightly shaken, incubated at 37° C. and 5% CO₂. Cultured for 4-6 h, the culture medium was replaced with DMEM containing 10% fetal calf serum.

Transfection efficiency (ie, the proportion of cells with green fluorescence) was observed the next day of the transfection, and ˜80% positive transfection efficiency indicated successful transfection experiment. After 48 h or 72 h of transfection, a 0.45 μm filter (purchased from Millipore Company) was used to filter and collect the virus, and centrifuged by Beckman Optima L-100 XP ultracentrifuge at 28,000 rpm, 4° C. for 2 hours, and the centrifuged supernatant was discarded. The precipitate obtained by centrifugation was resuspended with Quantum 007 culture solution (purchased from PAA Company) at 1/10-1/50 volume of the stock solution, packed at 100 μL/pipe and frozen at −80° C., waiting for virus titration or infecting T lymphocyte.

5. Determination of Titer of the Lentivirus Packed with Mock or eGFP-F2A-CAR

At Day 1, 293T cells were inoculated to a 96-well culture plate at 1*10⁵/ml, with 100 μL/well, incubated at 37° C. and 5% CO₂. The culture medium is DMEM medium containing 10% fetal calf serum. At day 2, 50 L/well culture supernatant was discarded, and 50 L/well fresh culture medium was added, which contains polybrene at a final concentration of 6 μg/ml, and incubated at 37° C., 5% CO₂ for 30 min. 10 μL/well of virus stock solution or 1 μL/well of concentrated virus solution was added, diluted for 5 times, with 4 gradients, in duplicate. Incubated at 37° C. 5% CO₂ After 48 hours of infection, the flow cytometry was used for detecting eGFP preferably with positive rate at 5-20% of the cell number, calculated as titer (U/ml)=positive efficiency×dilution times×100×10⁴. The titers of the above mentioned viruses packaged by the calcium phosphate transfection method, comprising MOCK (i.e. the empty vector control) and each eGFP-F2A-CAR were all at about 0.5-2*10⁶ U/mL. Virus titer measured after concentration was about 2×10⁷ U/mL.

### Example 4 Recombinant Lentivirus Infection of CTL Cells

Human peripheral blood mononuclear cells (provided by Shanghai Blood Center) were obtained from healthy human peripheral blood through density gradient centrifugation method. CTL cells were obtained from peripheral blood mononuclear cells by negative sorting method with CTL cell magnetic beads (purchased from Stem Cell Technologies), and the sorted CTL cells were subjected to flow cytometry to determine the purity of the CTL cells. CTL positive rate≥95% was preferred for next operation. Quantum 007 lymphocyte culture medium (purchased from PAA company) was added at a density of about 1×10⁶/ml for culturing, and magnetic beads (Invitrogen company) coated with both anti-CD3 and CD28 antibodies, at 1:1 cell:magnetic bead ratio, and recombinant human IL-2 with a final concentration of 100 U/ml (purchased from Shanghai Huaxin High Biotechnology Inc.) were added for stimulating and culturing for 24 h. Then the above recombinant lentivirus were used to infect the CTL cells at MOI≈5. The infected cells were subjected to passage at a density of 5*10⁵/ml every other day, while recombinant human IL-2 with a final concentration of 100 U/ml was supplemented to the lymphocyte culture medium.

At the 8^{th} day of culture, each different chimeric antigen receptor expression in infected CTL cells was tested by flow cytometry. Because eGFP and CAR were co-expressed, the cells detected to be eGFP positive were the positive cells expressing chimeric antigen receptors. Non-infected T lymphocytes were taken as negative control, and the positive rate of the virus expressing different chimeric antigen receptors infecting CTL cells were shown in table 3. The positive rate results showed that lentivirus infection method can obtain CAR CTL cells with certain positive rate.

TABLE 3

| CTL cells transfected with the following | eGFP possitive rate |
|---|---|
| CAR | of the CTL cells |
| Mock(empty vector control) | 56% |
| CLD18A2-Z, fused and expressing 163 single | 51% |
| chain antibody | |
| CLD18A2-28BBZ, fused and expressing 163 | 54% |
| single chain antibody | |
| CLD18A2-Z, fused and expressing 175 single | 52% |
| chain antibody | |
| CLD18A2-28BBZ, fused and expressing 175 | 55% |
| single chain antibody | |

After respectively infected and packaged with different chimeric antigen receptors, the CTL cells were passaged at cell density of 5*10⁵/ml and counted, and IL-2 (final concentration of 100 U/ml) was added to the passage cell culture solution. At 11^{th} day of culture, there is amplification of about 20-40 times, indicating that the CTL cells expressing different chimeric antigen receptors have the ability to amplify in vitro, thus ensuring subsequent in-vitro toxicity tests and in-vivo experiments.

### Example 5 In Vitro Toxicity Effect Assay for the Cells Expressing Chimeric Antigen Receptor

The materials used in the in-vitro toxicity experiment are as follows:

The 293T and gastric cancer cell lines as shown in table 4 were used as target cells. The effector cells were CTLs that were in vitro cultured for 12 days as verified in example 4, and confirmed by FACS that they were chimeric antigen receptor expression positive (noted as CAR⁺, chimeric antigen receptor positive). The effect:target ratios, upon different conditions were 3:1, 1:1, and 1:3, the number of the target cells was 10000/well. According to different effect:target ratio, each group set for five repeated wells, and the average value in 5 repeated wells was taken into count. The detection time was the 18th hour.

Wherein each experiment group and each control group were as follows:
Each experiment group: Each target cell+CTL expressing different chimeric antigen receptors,
Control group 1: target cell with maximum release of LDH
Control group 2: Target cell with spontaneous release of LDH
Control group 3: effector cells with spontaneous release of LDH.

Detection method: Carried out with CytoTox 96 non-radioactive cytotoxicity assay kit (Promega company). The method was a detection method based on colorimetric method, and can replace ⁵¹Cr release assay. CytoTox 96® assay quantitatively determines lactate dehydrogenase (LDH). LDH is a stable cytoplasmic enzyme, is released during cell lysis, whose release profile is substantially the same as the release profile of ⁵¹Cr in radioactivity analysis. The released LDH would be in the culture supernatant and can be detected by a 30-minute coupled enzyme reaction. In enzyme reaction, LDH can transfer a tetrazole salt (INT) into red Formazan. The red product is directly proportional to the number of lyzed cells. Refer to the instruction of CytoTox 96 non-radioactive cytotoxicity assay kit for details.

The cytotoxicity calculation formula is as follows:

$$cytotoxicity \% = \frac{Experimental group - Control group 2 - Control group 3}{Control group 1 - Control group 2} \times 100 \%$$

As specifically shown in table 4 and table 5, the present CTLs expressing chimeric antigen receptor (fusion expressing single chain antibody 163 or 175) CLD18A2-Z CAR⁺ and CLD18A2-28BBZ CAR⁺ have significant killing effect on 293T cells with high expression of CLD18A2, but not on the 293T cells expressing CLD18A1, which indicates that they can selectively kill the cells with CLD18A2. Furthermore, the present CTLs expressing chimeric antigen receptor CLD18A2-Z CAR⁺ and CLD18A2-28BBZ CAR⁺ have also significant killing effect on two gastric cancer cell lines BGC-823 and NCI-N87 with high expression of CLD18A2 (see table 4 and table 5), and it showed effect:target ratio dependency, that is, the higher the effect:target ratio, the stronger the cytotoxicity. However, there was no cytotoxicity to BGC-823 and NCI-N87 that do not express CLD18A2.

The data of the effect-target dependency further indicated that the present CTL of anti-CLD18A2 chimeric antigen receptor showed specific cytotoxicity to gastric cancer cells with high CLD18A2 expression.

Comparatively, the CTL transfected by MOCK plasmid (empty plasmid vector pWPT-eGFP not carrying CLD18A2-CAR) showed quite low cytotoxicity to above 3 cell lines with high CLD18A2 expression. The data of cytotoxicity to cell lines with high CLD18A2 expression exhibits significant difference between the CTL transfected by MOCK plasmid and the CTL expressing the present anti-CLD18A2 chimeric antigen receptor.

The above results showed that the chimeric antigen receptor constructed by choosing the single-chain antibody against CLD18A2 can selectively kill target cells with high CLD18A2 expression. In addition, from the cytotoxicity data, CAR T of CLD18A2-28BBZ has stronger cytotoxicity to cells expressing CLD18A2 than CART of CLD18A2-Z.

TABLE 4
Cytotoxicity of CAR T cells expressing single-chain antibody 163

| | CLD18A2-28BBZ | | | CLD18A2-Z | | | MOCK | | |
|---|---|---|---|---|---|---|---|---|---|
| | different | | | different | | | different | | |
| | effect:target ratio | | | effect:target ratio | | | effect:target ratio | | |
| CYTOTOXICITY (%) | 3:1 | 1:1 | 1:3 | 3:1 | 1:1 | 1:3 | 3:1 | 1:1 | 1:3 |
| 293T-CLD18A1 | 8.9 | 7.3 | 6.2 | 5.2 | 4.8 | 4.3 | 3.1 | 2.3 | 2.6 |
| 293T-CLD18A2 | 50 | 38.9 | 16.7 | 30.4 | 20.8 | 13.6 | 5.4 | 4.4 | 4.5 |
| BGC-823-CLD18A2 | 62.7 | 44.7 | 16.1 | 38.9 | 25.8 | 10.5 | 5.7 | 4.8 | 4.3 |
| BGC-823 | 5.8 | 5.5 | 4.8 | 5.2 | 4.6 | 5.5 | 2.8 | 3.5 | 3.8 |
| NCI-N87-CLD18A2 | 61.3 | 52.5 | 13.6 | 42.8 | 26.3 | 7.7 | 6.1 | 5.2 | 4.7 |
| NCI-N87 | 4.6 | 5.1 | 5.9 | 3.6 | 3.5 | 4.5 | 3.9 | 4.2 | 3.1 |

TABLE 5
Cytotoxicity of CAR T cells expressing single-chain antibody 175

| | CLD18A2-28BBZ | | | CLD18A2-Z | | | mock | | |
|---|---|---|---|---|---|---|---|---|---|
| | different | | | different | | | different | | |
| | effect:target ratio | | | effect:target ratio | | | effect:target ratio | | |
| cytotoxicity (%) | 3:1 | 1:1 | 1:3 | 3:1 | 1:1 | 1:3 | 3:1 | 1:1 | 1:3 |
| 293T-CLD18A1 | 8.6 | 7.3 | 6 | 5.4 | 4.8 | 2.7 | 2.4 | 1.5 | 1.4 |
| 293T-CLD18A2 | 57.5 | 41 | 15 | 35.2 | 25.7 | 13.2 | 5.6 | 2.3 | 1.6 |
| BGC-823-CLD18A2 | 69.4 | 45.4 | 17.2 | 43.5 | 24.6 | 8.7 | 9.2 | 6.9 | 3.4 |
| BGC-823 | 4.5 | 4.8 | 5.2 | 3.8 | 3.9 | 4.5 | 2.5 | 3.5 | 2.8 |
| NCI-N87-CLD18A2 | 68.2 | 44.2 | 16.3 | 41.5 | 28.2 | 10.2 | 10.1 | 8.2 | 3.3 |
| NCI-N87 | 5.2 | 2.9 | 3.9 | 4.2 | 3.3 | 4.5 | 2.5 | 3.2 | 4.3 |

### DISCUSSION

At present, CAR T cells have become a potential therapeutic means. However, many tumors, such as gastric cancer, do not have reports about CAR T cell therapy. There is research showing that CLD18A2 may be a specific marker of stomach tissue, therefore, it can also be a therapeutic target for tumor such as stomach cancer. However, at present, only the monoclonal antibody has been considered as the candidate drug with CLD18A2 as the therapeutic target, and whether it can be successfully used for the corresponding tumor treatment or not is not yet known. Therefore, it is necessary to find new treatment means. Considering the tissue specificity of CLD18A2, the present invention contemplates that if targeted therapy can be performed with CAR T cells, a novel antitumor formulation can be expected. However, it is known that the CLD18A2 antigen is a tightly-connected protein, whether it can contact with CAR T cells and induce the killing of the corresponding target cells is not known. In addition, as the protein spatial conformation is very crucial to the whole protein, many monoclonal antibody lose their antigen binding activity or specificity when evolving into single-chain antibodies. Fortunately, the inventors found that two single-chain antibodies (163 and 175) retained the antigen binding specificity of the monoclonal antibody. Further research shows that the CAR T cells composed of these two single-chain antibodies retained the selective killing effect on CLD18A2 positive cells. The results of the invention showed that CLD18A2 can truly be a CAR T cell therapy target; the CAR T cell against CLD18A2 is a novel candidate tumor treatment candidate means.

All documents mentioned in this disclosure are all incorporated herein by reference, as if each document is individually referred to as a reference. Furthermore, it should be understood that after reading the above teachings of the invention, those skilled in the art can make various variations or modifications to the present invention, these equivalent forms are also within the scope defined by the appended claims.

1-17. (canceled)

18. A chimeric antigen receptor (CAR) for inducing cytotoxic effect against CLD18A2 positive cells, comprising an extracellular binding region, a transmembrane region, and an intracellular signal region, wherein the extracellular binding region specifically recognizes CLD18A2.

19. The CAR of claim 18, wherein the extracellular binding region comprises an antibody specifically recognizing CLD18A2 or a ligand specifically recognizing CLD18A2.

20. The CAR of claim 18, wherein the extracellular binding region comprises monoclonal antibody, a single-chain antibody, or a domain antibody specifically recognizing CLD18A2.

21. The CAR of claim 18, wherein the extracellular binding region comprises a heavy chain variable region and a light chain variable region.

22. The CAR of claim 18, wherein the intracellular signal region comprises intracellular signal activation motif.

23. The CAR of claim 18, wherein the intracellular signal region comprises one or more immunoreceptor tyrosine-based activation motif (ITAM).

24. The CAR of claim 18, wherein the intracellular signal region comprises an intracellular signal region of CD3ζ.

25. The CAR of claim 24, wherein the intracellular signal region further comprises an intracellular signal region of CD28, CD137, CD134, or a combination thereof.

26. The CAR of claim 18, wherein the transmembrane region comprises a transmembrane region of a natural marker on a surface of a T lymphocyte.

27. The CAR of claim 18, wherein the transmembrane region comprises a transmembrane region of CD8 or a transmembrane region of CD28.

28. The CAR of claim 18, wherein the transmembrane region comprises a transmembrane region of CD8 alpha.

29. The CAR of claim 18, wherein the transmembrane region further comprises a hinge region.

30. The CAR of claim 29, wherein the hinge region comprises a hinge region of CD8 or a hinge region of CD28.

31. The CAR of claim 18, wherein the CAR is connected in an order of:
(i) the scFv specifically recognizing CLD18A2, a CD8α hinge region, a CD8 transmembrane region, and a CD3ζ intracellular signal region;
(ii) the scFv specifically recognizing CLD18A2, a CD8α hinge region, a CD8 transmembrane region, a CD137 intracellular signal region, and a CD3ζ intracellular signal region;
(iii) the scFv specifically recognizing CLD18A2, a CD8α hinge region, a CD28 transmembrane region, a CD28 intracellular signal region, and a CD3ζ intracellular signal region; or
(iv) the scFv specifically recognizing CLD18A2, a CD8α hinge region, a CD28 transmembrane region, a CD28 intracellular signal region, a CD137 intracellular signal region, and the CD3ζ intracellular signal region.

32. The CAR of claim 31, wherein the CD8α hinge region comprises the CD8α hinge region in SEQ ID NO: 20, wherein the CD8 transmembrane region comprises the CD8 transmembrane region in SEQ ID NO: 19, wherein the CD28 transmembrane region comprises the CD28 transmembrane region in SEQ ID NO: 20, wherein the CD28 intracellular signal region comprises the CD28 intracellular signal region in SEQ ID NO: 20, wherein the CD137 intracellular signal region comprises the CD137 intracellular signal region in SEQ ID NO: 20, and wherein the CD3ζ intracellular signal region comprises the CD3ζ intracellular signal region in SEQ ID NO: 20.

33. The CAR of claim 18, further comprising a signal peptide.

34. An immune effector cell expressing the CAR of claim 18.

35. The immune effector cell of claim 34, wherein the immune effector cell comprises T lymphocytes, NK cells or NKT cells.

36. A method of treating pancreatic cancer or stomach cancer, wherein the method comprises administering to a subject in need thereof an effective amount of immune effector cells, wherein the immune effector cells comprise the chimeric antigen receptor (CAR) for inducing cytotoxic effect against CLD18A2 positive cells, comprising an extracellular binding region, a transmembrane region, and an intracellular signal region, wherein the extracellular binding region specifically recognizing CLD18A2.


SEQUENCE LISTING (26 sequences)

SEQ ID NO: 1 - DNA, 727 bp, synthetic construct
  source 1..727: mol_type = other DNA; note = Nucleic acid sequence encoding CLD18A2 single chain antibody 125
gctagcacaggttcagctgcagcagtctggagctgagctggcgaggcccggggcttcagtgaagctgtcctgcaaggcttctggctacaccttcactgactactatataaactgggtgaagcagaggactggacagggccttgagtggattggagagatttatcctggaagtggtaatacttactacaatgagaagttcaagggcaaggccacactgactgcagacaaatcctccagcacagcctacatgcagctcagcagcctgacatctgaggactctgcagtctatttctgtgcaagatcgtatggtgcctttgactactggggccaaggcaccactctcacagtctcctcaggtggaggcggttcaggcggaggtggctctggcggtggcggatcggacattgtgatgacccagtctcaaaaattcatgtccacatcagtaggagacagggtcagcatcacctgcaaggccagtcagaatgttcgtactgctgtagcctggtatcaacagaaaccagggcagtctcctaaagcactgatttacttggcatccaaccggcacactggagtccctgatcgcttcacaggcagtggatctgggacagatttcactctcaccattagcaatgtgcaatctgaagacctggcagattatttctgtctgcaacattggaattatcctctgacgttcggtggaggcaccaagctggaaatcaaaggatcc

SEQ ID NO: 2 - protein, 238 aa, synthetic construct
  source 1..238: note = Amino acid sequence of CLD18A2 single chain antibody 125
QVQLQQSGAELARPGASVKLSCKASGYTFTDYYINWVKQRTGQGLEWIGEIYPGSGNTYYNEKFKGKATLTADKSSSTAYMQLSSLTSEDSAVYFCARSYGAFDYWGQGTTLTVSSGGGGSGGGGSGGGGSDIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGGGTKLEIK

SEQ ID NO: 3 - DNA, 751 bp, synthetic construct
  source 1..751: mol_type = other DNA; note = Nucleic acid sequence encoding CLD18A2 single chain antibody 165
gctagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacctatgctgaagagttcaagggacggtttgccttctctttggaaacctctgccagcactgcctatttgcagatcaacaacctcaaaaatgaggacacggctacatatttctgtgcaagactgggttttggtaatgctatggactactggggtcaaggaacctcagtcaccgtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaagaattc

SEQ ID NO: 4 - protein, 246 aa, synthetic construct
  source 1..246: note = Amino acid sequence of CLD18A2 single chain antibody 163
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 5 - DNA, 751 bp, synthetic construct
  source 1..751: mol_type = other DNA; note = Nucleic acid sequence encoding CLD18A2 single chain antibody 175
gctagcacaggtccaactgcagcagcctggggctgagctggtgaggcctggggcttcagtgaagctgtcctgcaaggcttctggctacaccttcaccagctactggataaactgggtgaagcagaggcctggacaaggccttgagtggatcggaaatatttatccttctgatagttatactaactacaatcaaaagttcaaggacaaggccacattgactgtagacaaatcctccagcacagcctacatgcagctcagcagcccgacatctgaggactctgcggtctattactgtacaagatcgtggaggggtaactcctttgactactggggccaaggcaccactctcacagtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaagaattc

SEQ ID NO: 6 - protein, 246 aa, synthetic construct
  source 1..246: note = Amino acid sequence of CLD18A2 single chain antibody 175
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 7 - DNA, 42 bp, synthetic construct
  source 1..42: mol_type = other DNA; note = Primer
tgctccacgccgccaggccgcagatccagttggtgcagtctg

SEQ ID NO: 8 - DNA, 42 bp, synthetic construct
  source 1..42: mol_type = other DNA; note = Primer
cgcggcgctggcgtcgtggttttcagctccagcttggtccca

SEQ ID NO: 9 - DNA, 40 bp, synthetic construct
  source 1..40: mol_type = other DNA; note = Primer
tgctccacgccgccaggccgcaggtccaactgcagcagcc

SEQ ID NO: 10 - DNA, 43 bp, synthetic construct
  source 1..43: mol_type = other DNA; note = Primer
cgcggcgctggcgtcgtggtttttatttccaactttgtccccg

SEQ ID NO: 11 - DNA, 22 bp, synthetic construct
  source 1..22: mol_type = other DNA; note = Primer
gcaggggaaagaatagtagaca

SEQ ID NO: 12 - DNA, 20 bp, synthetic construct
  source 1..20: mol_type = other DNA; note = Primer
cggcctggcggcgtggagca

SEQ ID NO: 13 - DNA, 20 bp, synthetic construct
  source 1..20: mol_type = other DNA; note = Primer
accacgacgccagcgccgcg

SEQ ID NO: 14 - DNA, 22 bp, synthetic construct
  source 1..22: mol_type = other DNA; note = Primer
tagcgtaaaaggagcaacatag

SEQ ID NO: 15 - DNA, 2183 bp, synthetic construct
  source 1..2183: mol_type = other DNA; note = Nucleic acid sequence encoding chimeric antigen receptor protein CLD18A2-163-Z
aagcttacgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatggccttaccagtgaccgccttgctcctgccgctggccttgctgctccacgccgccaggccgcagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacctatgctgaagagttcaagggacggtttgccttctctttggaaacctctgccagcactgcctatttgcagatcaacaacctcaaaaatgaggacacggctacatatttctgtgcaagactgggttttggtaatgctatggactactggggtcaaggaacctcagtcaccgtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacctcgagggaattc

SEQ ID NO: 16 - DNA, 2450 bp, synthetic construct
  source 1..2450: mol_type = other DNA; note = Nucleic acid sequence encoding chimeric antigen receptor protein CLD18A2-163-28BBZ
aagcttacgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatggccttaccagtgaccgccttgctcctgccgctggccttgctgctccacgccgccaggccgcagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacctatgctgaagagttcaagggacggtttgccttctctttggaaacctctgccagcactgcctatttgcagatcaacaacctcaaaaatgaggacacggctacatatttctgtgcaagactgggttttggtaatgctatggactactggggtcaaggaacctcagtcaccgtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacctcgagggaattc

SEQ ID NO: 17 - DNA, 2183 bp, synthetic construct
  source 1..2183: mol_type = other DNA; note = Nucleic acid sequence encoding chimeric antigen receptor protein CLD18A2-175-Z
aagcttacgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatggccttaccagtgaccgccttgctcctgccgctggccttgctgctccacgccgccaggccgcaggtccaactgcagcagcctggggctgagctggtgaggcctggggcttcagtgaagctgtcctgcaaggcttctggctacaccttcaccagctactggataaactgggtgaagcagaggcctggacaaggccttgagtggatcggaaatatttatccttctgatagttatactaactacaatcaaaagttcaaggacaaggccacattgactgtagacaaatcctccagcacagcctacatgcagctcagcagcccgacatctgaggactctgcggtctattactgtacaagatcgtggaggggtaactcctttgactactggggccaaggcaccactctcacagtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacctcgagggaattc

SEQ ID NO: 18 - DNA, 2450 bp, synthetic construct
  source 1..2450: mol_type = other DNA; note = Nucleic acid sequence encoding chimeric antigen receptor protein CLD18A2-175-28BBZ
aagcttacgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagtgaaacagactttgaattttgaccttctgaagttggcaggagacgttgagtccaaccctgggcccatggccttaccagtgaccgccttgctcctgccgctggccttgctgctccacgccgccaggccgcaggtccaactgcagcagcctggggctgagctggtgaggcctggggcttcagtgaagctgtcctgcaaggcttctggctacaccttcaccagctactggataaactgggtgaagcagaggcctggacaaggccttgagtggatcggaaatatttatccttctgatagttatactaactacaatcaaaagttcaaggacaaggccacattgactgtagacaaatcctccagcacagcctacatgcagctcagcagcccgacatctgaggactctgcggtctattactgtacaagatcgtggaggggtaactcctttgactactggggccaaggcaccactctcacagtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacctcgagggaattc

SEQ ID NO: 19 - protein, 709 aa, synthetic construct
  source 1..709: note = Chimeric antigen receptor protein CLD18A2-163-Z
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMALPVTALLLPLALLLHAARPQIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 798 aa, synthetic construct
  source 1..798: note = Amino acid sequence of chimeric antigen receptor protein CLD18A2-163-28BBZ
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMALPVTALLLPLALLLHAARPQIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 709 aa, synthetic construct
  source 1..709: note = Amino acid sequence of chimeric antigen receptor protein CLD18A2-175-Z
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMALPVTALLLPLALLLHAARPQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 798 aa, synthetic construct
  source 1..798: note = Amino acid sequence of chimeric antigen receptor protein CLD18A2-175-28BBZ
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGVKQTLNFDLLKLAGDVESNPGPMALPVTALLLPLALLLHAARPQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - DNA, 1266 bp, synthetic construct
  source 1..1266: mol_type = other DNA; note = scFv(GPC3)-CD8-CD3 zeta
gatgttgtgatgactcagtctccactctccctgcccgtcacccctggagagccggcctccatctcctgcagatctagtcagagccttgtacacagtaatgccaacacctatttacattggtacctgcagaagccagggcagtctccacagctcctgatctataaagtttccaaccgattttctggggtccctgacaggttcagtggcagtggatcaggcacagattttacactgaaaatcagcagagtggaggctgaggatgttggggtttattactgctctcaaaatacacatgttcctcctacgtttggccaggggaccaagctggagatcaaacgtggtggaggcggttcaggcggaggtggctctggcggtggcggatcgcaggtgcagctggtgcagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacgaatccacgagcacagcctacatggagctgagcagcctgagatctgaggacacggccgtgtattactgtacaagattctactcctatacttactggggccagggaaccctggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 24 - DNA, 1533 bp, synthetic construct
  source 1..1533: mol_type = other DNA; note = scFv(GPC3)-CD28a-CD28b-CD137-CD3 zeta
gatgttgtgatgactcagtctccactctccctgcccgtcacccctggagagccggcctccatctcctgcagatctagtcagagccttgtacacagtaatgccaacacctatttacattggtacctgcagaagccagggcagtctccacagctcctgatctataaagtttccaaccgattttctggggtccctgacaggttcagtggcagtggatcaggcacagattttacactgaaaatcagcagagtggaggctgaggatgttggggtttattactgctctcaaaatacacatgttcctcctacgtttggccaggggaccaagctggagatcaaacgtggtggaggcggttcaggcggaggtggctctggcggtggcggatcgcaggtgcagctggtgcagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacgaatccacgagcacagcctacatggagctgagcagcctgagatctgaggacacggccgtgtattactgtacaagattctactcctatacttactggggccagggaaccctggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 25 - protein, 7 aa, synthetic construct
DYKDDDK

SEQ ID NO: 26 - protein, 15 aa, synthetic construct
GGGGSGGGGSGGGGS